(19) United States
(12) Patent Application Publication
(10) Pub. No.: US 2026/0108032 A1
(43) Pub. Date: Apr. 23, 2026

(54) Case for Oral Hygiene Accessories

(71) Applicant: ANDRES GIRO TEIXIDO, Santo Domingo Este (DO)

(72) Inventor: ANDRES GIRO TEIXIDO, Santo Domingo Este (DO)

(21) Appl. No.: 19/361,527

(22) Filed: Oct. 17, 2025

(30) Foreign Application Priority Data
Oct. 18, 2024 (ES) 202431930

(51) Int. Cl. A45C 11/00; A45C 13/02; A45C 15/04; A45C 15/06; A61C 15/04; A61C 19/02; A61L 2/10; A61L 2/24

(52) U.S. Cl. CPC A45C 11/008; A45C 13/02; A45C 15/04; A45C 15/06; A61C 15/046; A61C 19/02; A61L 2/10; A61L 2/24; A61L 2202/11; A61L 2202/121; A61L 2202/122; A61L 2202/123; A61L 2202/14; A61L 2202/16

(57) ABSTRACT

A case for oral hygiene accessories the case including a casing consisting of two portions joined together, one portion being a lid and a second portion being a base, the casing defining a front part and a rear part. One of the two portions has an internal geometry that defines two housings separated by a dividing section, the housings being sized to house an oral hygiene accessory in each of them. The oral hygiene accessory including a single body without a handle that comprises a portion of dental floss supported at two ends by two extensions facing each other.

### CROSS-REFERENCE TO RELATED APPLICATIONS

This is a Non-Provisional Application which claims priority to Spanish Application No. 202431930 filed Oct. 18, 2024. The disclosure of the prior application is hereby incorporated by reference in its entirety.

### FIELD OF THE INVENTION

The present invention falls within the field of oral hygiene accessories, in particular, devices that facilitate the use and handling of products such as dental floss, incorporating features that improve hygiene and user comfort. More specifically, the invention relates to an improved case configured to store and protect single-person oral hygiene accessories, the accessory being of the type comprising a small-sized part fitted with dental floss.

### BACKGROUND OF THE INVENTION

Traditional cases for storing dental floss and other oral hygiene accessories exist, but they often lack ergonomic features and disinfection systems, which limits their effectiveness in terms of hygiene.

Some oral hygiene devices incorporate mirrors to make it easier to check teeth. However, they often do not incorporate disinfection systems or a design that allows for hands-free use.

There are personal care cases that have an ergonomic design.

However, these cases are often intended for other types of products and do not address specific oral hygiene needs.

Therefore, there is still a need to develop a case that overcomes the aforementioned drawbacks.

Furthermore, the applicant is currently unaware of any invention that has all of the features described in this specification.

### SUMMARY OF THE DISCLOSURE

The present invention has been developed with the aim of providing an improved case that is configured as a novelty within the field of application and solves the previously mentioned drawbacks, further contributing other additional advantages that will be obvious from the description below.

Therefore, an object of the present invention is to provide an improved case provided for the protected storage of oral hygiene accessories, in particular, an accessory consisting of a single body without a handle that includes a portion of dental floss supported at two ends by means of two extensions facing each other, wherein the single body has dimensions configured to be inserted into an oral cavity.

In particular, the invention is characterised in that it comprises a casing consisting of two portions joined together, one portion being a lid and a second portion being a base, the casing defining a front part and a rear part, wherein one of the two portions has an internal geometry that defines two housings separated by a dividing section, the housings being sized to house an oral hygiene accessory in each of them.

According to another aspect of the invention, the rim defining the set of the two housings is inclined from the rear part towards the front part of the casing, so that each of the housings has a rear wall that is higher than a front wall.

Preferably, the two portions are joined at the rear part by means of a hinged portion.

Advantageously, the lid portion includes an inner mirror, allowing the user to observe possible residue on their teeth, enabling a quick and effective check of the oral condition.

According to another aspect of the invention, the case incorporates an ultraviolet light disinfection system, which can be activated or deactivated by the user, thus contributing to the elimination of germs and bacteria, guaranteeing a better hygiene.

Preferably, the disinfection system includes a timer configured to determine a pre-set time period of operation, ensuring practical and efficient use of the device.

Advantageously, the casing has a recharging port provided for recharging a battery that provides an electric current to a control board that manages the disinfection system, allowing prolonged and convenient use of the disinfection system.

Also advantageously, the front part of the casing includes a protrusion protruding from the top of the base portion that is sized to fit into a recess in the front part of the lid portion. This makes it easier to open and close the lid, improving user experience.

Preferably, the disinfection system has two ultraviolet light sources, each of which is directed at a respective housing.

According to the invention, the casing is made of a rigid injection mouldable plastic material.

In some aspects, the techniques described herein relate to a case for oral hygiene accessories, provided for storing at least one oral hygiene accessory, the case including: a casing including two portions joined together, one portion being a lid and a second portion being a base, the casing defining a front part and a rear part, wherein one of the two portions has an internal geometry that defines two housings separated by a dividing section, each housings being sized to house the oral hygiene accessory in each housing; and a rim defining the two housings is inclined from the rear part towards the front part of the casing, such that each of the housings has a rear wall that is higher than a front wall.

In some aspects, the techniques described herein relate to a case, wherein the two portions are joined at the rear part by a hinged portion.

In some aspects, the techniques described herein relate to a case, further including an inner mirror in the lid.

In some aspects, the techniques described herein relate to a case, further including an ultraviolet light disinfection system, which can be activated or deactivated by a user via a push button.

In some aspects, the techniques described herein relate to a case, wherein the disinfection system further includes a timer configured to determine a pre-set time period of operation of the disinfection system.

In some aspects, the techniques described herein relate to a case, further including: a recharging port provided for recharging a battery that provides an electric current to a control board that manages the disinfection system.

In some aspects, the techniques described herein relate to a case, wherein the front part of the casing includes a protrusion protruding upward of the base portion that is sized to fit into a recess in the front part of the lid portion.

In some aspects, the techniques described herein relate to a case, wherein the disinfection system has two ultraviolet light sources, each of light source being directed at a respective housing.

In some aspects, the techniques described herein relate to a case, characterised in that the casing is made of a rigid injection mouldable plastic material.

In some aspects, the techniques described herein relate to a case, wherein the oral hygiene accessory further includes: a single body without a handle that includes a portion of dental floss supported at two ends by means of two extensions facing each other, wherein the single body has dimensions configured to be inserted into an oral cavity.

These features allow for efficient and organised storage of oral hygiene accessories, so that the user has a physical place to store the accessory that can be used on one or more occasions.

The disclosed case offers a comprehensive solution for oral hygiene, combining convenience, efficiency and a focus on personal hygiene. Its innovative design not only enhances user experience, but also promotes healthier dental care practices, reasons that give it sufficient grounds for having the privilege of exclusivity being requested.

Other features and advantages of the improved case object of the present invention will become apparent from the description of a preferred but not exclusive embodiment illustrated by way of non-limiting example in the attached drawings, in which:

### BRIEF DESCRIPTION OF THE DRAWINGS

Other features and advantages of the improved case object of the present invention will become apparent from the description of a preferred but not exclusive embodiment illustrated by way of non-limiting example in the attached drawings, in which:

FIG. 1 is a perspective view of an improved case according to the invention, wherein the casing is in an open condition;

FIG. 2 is a perspective view, seen from the side, of the improved case in an open condition;

FIG. 3 is a perspective view of an improved case according to the invention, wherein the casing is in a closed condition;

FIG. 4 is a perspective view of an improved case according to the invention, wherein the casing is in an open condition, incorporating an oral hygiene accessory in one of its housings;

FIG. 5 is a side view of improved case shown in above figures;

FIG. 6 is a side view of improved case from an opposed side according to side shown in FIG. 5;

FIG. 7 is a perspective view of an embodiment of a dental floss to be stored inside case of the invention; and

FIG. 8 is a perspective view of dental floss shown in FIG. 7 inserted into an oral cavity.

### DETAILED DESCRIPTION OF THE DISCLOSURE

In view of the aforementioned figures, and in accordance with the adopted numbering, a preferred exemplary embodiment of the invention is disclosed, which comprises the parts and elements indicated and described in detail below.

Moreover, the terms upper, lower, up, down, and the like in the description and in the claims are used for descriptive purposes and not necessarily to describe relative positions.

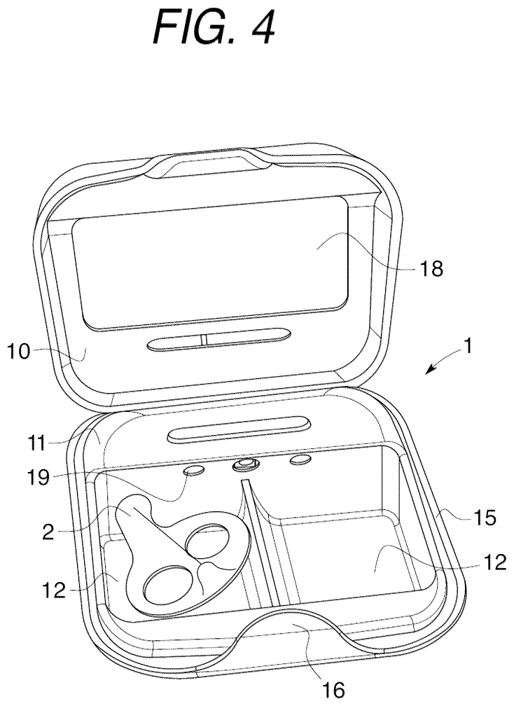

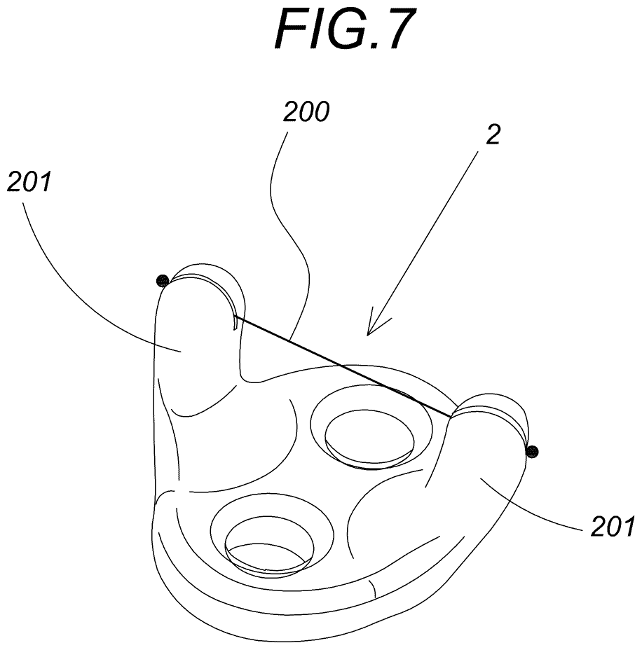

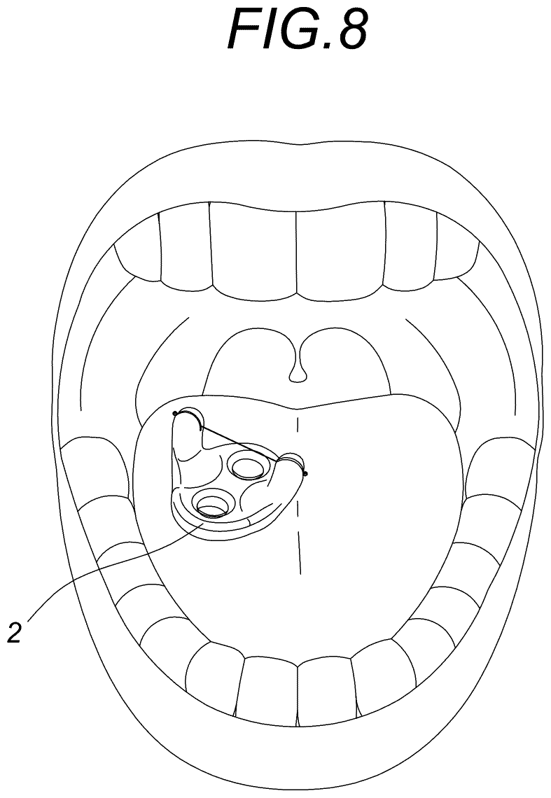

As can be seen in the attached figures, one embodiment of the case is designed and sized to store oral hygiene accessories, in particular, an oral hygiene accessory (2) (see FIG. 4) consisting of a single body without a handle that includes a portion of dental floss (200) supported at two ends by means of two extensions (201) facing each other (see FIG. 7), wherein the single body has dimensions configured to be inserted into an oral cavity (see FIG. 8).

The description of the oral hygiene accessory (2) will not be further disclosed herein as a more detailed description can be found in WO 2020212639 A which is hereby incorporated by reference in its entirety.

In more detail, the case, generally indicated by numeral reference (1), comprises a casing, made of a rigid injection mouldable plastic material, which includes two portions (10, 11) joined together, the first portion being a lid (10) and the second portion being a base (11).

The casing has a rectangular prismatic geometric shape with rounded corners, such that it defines a front part and a rear part, wherein the base portion (11) has an internal geometry that defines two housings (12) separated by a dividing section (13), each of the two housings (12) being sized to hold an oral hygiene accessory (2), as shown in FIG. 4.

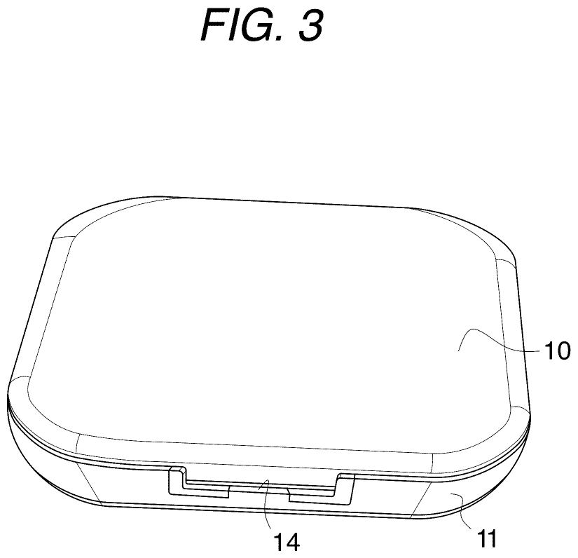

As shown in FIG. 3, the two portions (10, 11) are joined at the rear part by means of a hinged portion (14).

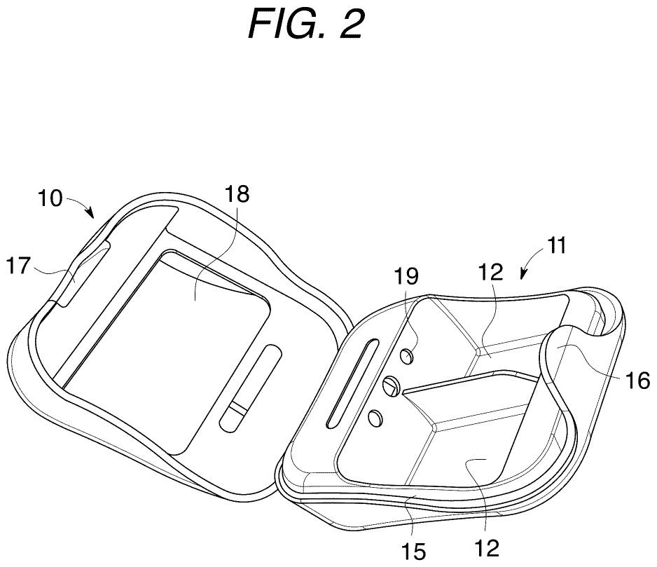

The rim defining the set of the two housings has an inclination (15) (see FIG. 2) from the rear part towards the front part of the casing, so that each of the housings (12) has a rear wall that is higher than a front wall.

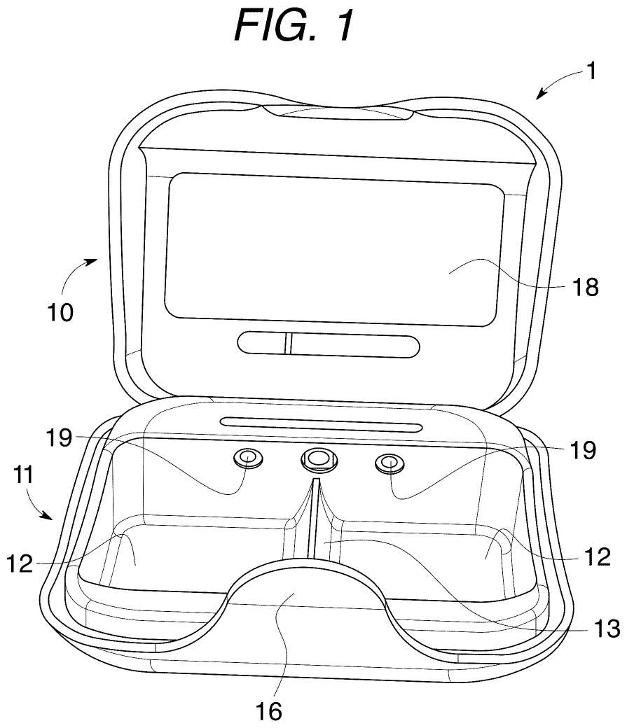

As can be seen in FIGS. 1, 2 and 4, the front part of the casing includes a curved protrusion (16) extending upwards from the base portion (11) that is sized to fit into a recess (17) in the front part of the lid portion (10).

Additionally, the lid portion (10) includes a mirror (18) mounted on its inner face.

To increase the degree of hygiene of the oral hygiene accessory (2), an ultraviolet light disinfection system (20) is provided, mounted inside the casing, which can be activated or deactivated by the user via a push button (23), including a timer (22) configured to determine a pre-set time period of operation.

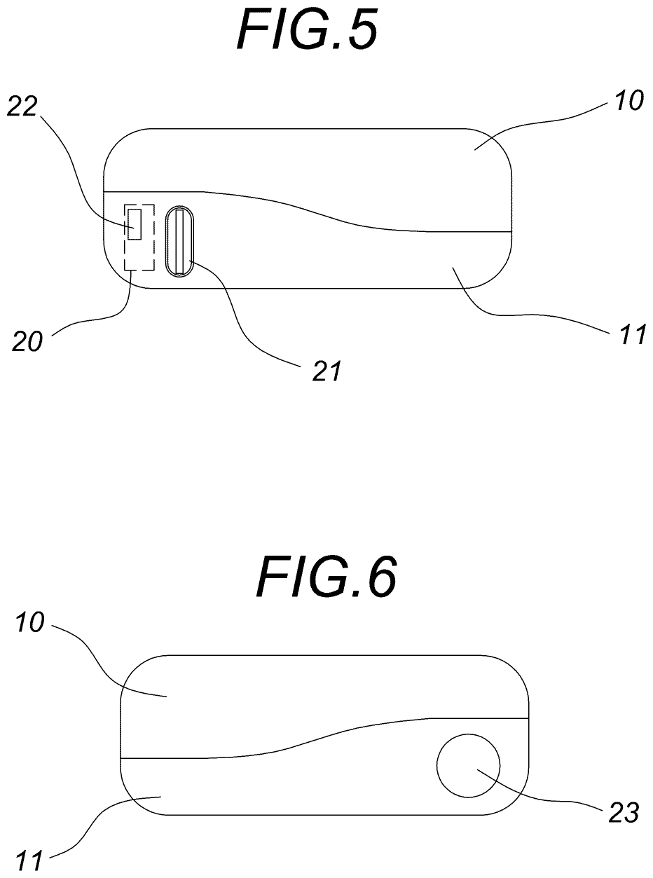

In order to recharge a battery that provides an electric current to a control board that manages the disinfection system, a recharging port (21), e.g., USB, is provided (see FIG. 5).

Referring again to the disinfection system, it has two ultraviolet light sources (19), each of which is directed at a respective housing (12).

The details, shapes, dimensions and other accessory elements used to manufacture the improved case of the invention may be suitably substituted for others that do not depart from the scope defined by the claims which are included below.

1. A case for oral hygiene accessories, provided for storing at least one oral hygiene accessory, the case comprising:
a casing including two portions joined together, one portion being a lid and a second portion being a base, the casing defining a front part and a rear part, wherein one of the two portions has an internal geometry that defines two housings separated by a dividing section, each housings being sized to house the oral hygiene accessory in each housing; and
a rim defining the two housings is inclined from the rear part towards the front part of the casing, such that each of the housings has a rear wall that is higher than a front wall.

2. The case according to claim 1, wherein the two portions are joined at the rear part by a hinged portion.

3. The case according to claim 1, further comprising an inner mirror in the lid.

4. The case according to claim 1, further comprising an ultraviolet light disinfection system, which can be activated or deactivated by a user via a push button.

5. The case according to claim 4, wherein the disinfection system further comprises a timer configured to determine a pre-set time period of operation of the disinfection system.

6. The case according to claim 4, further comprising:
a recharging port provided for recharging a battery that provides an electric current to a control board that manages the disinfection system.

7. The case according to claim 1, wherein the front part of the casing includes a protrusion protruding upward of the base portion that is sized to fit into a recess in the front part of the lid portion.

8. The case according to claim 4, wherein the disinfection system has two ultraviolet light sources, each of light source being directed at a respective housing.

9. The case according to claim 1, characterised in that the casing is made of a rigid injection mouldable plastic material.

10. The case according to claim 1, wherein the oral hygiene accessory further comprises:
a single body without a handle that comprises a portion of dental floss supported at two ends by means of two extensions facing each other, wherein the single body has dimensions configured to be inserted into an oral cavity.
